(19) United States
(12) Patent Application Publication
(10) Pub. No.: US 2022/0096962 A1
(43) Pub. Date: Mar. 31, 2022

(54) PROCESS FOR REMOVING LEAD, MERCURY, POTASSIUM, AND AMMONIUM IONS FROM BODILY FLUIDS USING RARE-EARTH SILICATE ION EXCHANGE COMPOSITIONS

(71) Applicant: UOP LLC, Des Plaines, IL (US)

(72) Inventors: Mimoza Sylejmani-Rekaliu, Bensenville, IL (US); James M. Hodges, Evanston, IL (US); Paulina Jakubczak, Elk Grove Village, IL (US); Evgeny Kolev, Arlington Heights, IL (US); Gregory J. Lewis, Santa Cruz, CA (US); William Sheets, Glenview, IL (US)

(21) Appl. No.: 17/406,331

(22) Filed: Aug. 19, 2021

Related U.S. Application Data
(60) Provisional application No. 63/085,804, filed on Sep. 30, 2020.

(51) Int. Cl. B01D 15/36; A61M 1/28; A61M 1/36; A61M 31/00; A61K 33/06; A61K 33/24; A61P 3/12; B01D 15/20; B01D 69/14; B01D 69/08; B01J 39/02; B01J 39/14; B01J 47/12

(52) U.S. Cl. CPC B01D 15/362; A61M 1/28; A61M 1/3687; A61M 31/002; A61K 33/06; A61K 33/24; B01J 47/12; B01D 15/206; B01D 69/147; B01D 69/08; B01J 39/02; B01J 39/14; A61P 3/12

(57) ABSTRACT

A process for removing Pb²⁺, Hg²⁺, K⁺ and NH₄⁺ toxins from bodily fluids is disclosed. The process involves contacting the bodily fluid with an ion exchange composition to remove the metal toxins in the bodily fluid, including blood and gastrointestinal fluid. Alternatively, blood can be contacted with a dialysis solution which is then contacted with the ion exchange composition. The ion exchange compositions are represented by the following empirical formula:

A^{r+}ₚM^{s+}₁₋ₓM′^{t+}ₓSiₙOₘ

A composition comprising the above ion exchange compositions in combination with bodily fluids or dialysis solution is also disclosed. The ion exchange compositions may be supported by porous networks of biocompatible polymers such as carbohydrates or proteins.

### CROSS-REFERENCE TO RELATED APPLICATIONS

This application claims priority from U.S. Provisional Application No. 63/085,804, filed Sep. 30, 2021, which is incorporated herein in its entirety.

### FIELD OF THE INVENTION

The present invention relates to intracorporeal and extracorporeal processes for removing heavy metal toxins, e.g. lead and mercury ions, and metabolic toxins, e.g., potassium and ammonium ions, from bodily fluids. The blood or other bodily fluid is placed in contact with a rare-earth silicate ion exchange composition that is capable of selectively removing the toxins. Alternatively, blood is first contacted with a dialysis solution that is then contacted with the rare-earth silicate ion exchange composition.

### BACKGROUND OF THE INVENTION

In mammals, e.g., humans, when the kidneys and/or liver fail to remove metabolic waste products from the body, most of the other organs of the body also soon fail. Accordingly, extensive efforts have been made to discover safe and effective methods for removing toxins from patients' blood by extracorporeal treatment of the blood. Many methods have been proposed for removing small molecular toxins, protein-bound molecules or larger molecules thought to be responsible for the coma and illness of hepatic failure. Some of these toxic compounds have been identified as urea, creatine, ammonia, phenols, mercaptans, short chain fatty acids, aromatic amino acids, false neural transmitters (octopamine), neural inhibitors (glutamate) and bile salts. The art shows a number of ways to treat blood containing such toxins. The classic method is of course, dialysis. Dialysis is defined as the removal of substances from a liquid by diffusion across a semipermeable membrane into a second liquid. Dialysis of blood outside of the body (hemodialysis) is the basis of the “artificial kidney.” The artificial kidney treatment procedure generally used today is similar to that developed by Kolff in the early 1940s. Since the 1940s there have been several disclosures which deal with improvements on artificial kidneys or artificial livers. Thus, U.S. Pat. No. 4,261,828 discloses an apparatus for the detoxification of blood. The apparatus comprises a housing filled with an adsorbent such as charcoal or a resin and optionally an enzyme carrier. In order to prevent direct contact between the blood and the adsorbent, the adsorbent may be coated with a coating which is permeable for the substances to be adsorbed yet prevent the direct contact between the corpuscular blood components and the adsorbents. U.S. Pat. No. 4,581,141 discloses a composition for use in dialysis which contains a surface adsorptive substance, water, a suspending agent, urease, a calcium-loaded cation exchanger, an aliphatic carboxylic acid resin and a metabolizable organic acid buffer. The calcium loaded cation exchanger can be a calcium-exchanged zeolite. EP 0046971 A1 discloses that zeolite W can be used in hemodialysis to remove ammonia. Finally, U.S. Pat. No. 5,536,412 discloses hemofiltration and plasma filtration devices in which blood flows through the interior of a hollow fiber membrane and during the flow of blood, a sorbent suspension is circulated against the exterior surfaces of the hollow fiber membrane. Another step involves having the plasma fraction of the blood alternately exit and re-enter the interior of the membrane thereby effectuating removal of toxins. The sorbent can be activated charcoal along with an ion-exchanger such as a zeolite or a cation-exchange resin.

There are problems associated with the adsorbents disclosed in the above patents. For example, charcoal does not remove any water, phosphate, sodium or other ions. Zeolites have the disadvantage that they can partially dissolve in the dialysis solution, allowing aluminum and/or silicon to enter the blood. Additionally, zeolites can adsorb sodium, calcium and potassium ions from the blood thereby requiring that these ions be added back into the blood.

More recently, examples of microporous ion exchangers that are essentially insoluble in fluids, such as bodily fluids (especially blood), have been developed, namely the zirconium-based silicates and titanium-based silicates of U.S. Pat. Nos. 5,888,472; 5,891,417 and 6,579,460. The use of these zirconium-based silicate or titanium-based silicate microporous ion exchangers to remove toxic ammonium cations from blood or dialysate is described in U.S. Pat. Nos. 6,814,871, 6,099,737, and 6,332,985. Additionally, it was found that some of these compositions were also selective in potassium ion exchange and could remove potassium ions from bodily fluids to treat the disease hyperkalemia, which is discussed in U.S. Pat. Nos. 8,802,152; 8,808,750; 8,877,255; 9,457,050; 9,662,352; 9,707,255; 9,844,567; 9,861, 658; 10,413,569; 10,398,730; US 2016/0038538 and U.S. Pat. No. 10,695,365. Ex-vivo applications of these materials, for instance in dialysis, are described in U.S. Pat. No. 9,943,637.

Blood compatible polymers have also been incorporated into devices for treating bodily fluids. U.S. Pat. No. 9,033,908 discloses small desktop and wearable devices for removing toxins from blood. The device features a sorption filter that utilizes nanoparticles embedded in a porous blood compatible polymeric matrix. Among the toxic materials targeted by this device and filter system are potassium, ammonia, phosphate, urea, and uric acid. Similarly, a 3-D printed hydrogel matrix consisting of crosslinked poly(ethylene glycol) diacrylate to which poly diacetylene-based nanoparticles are tethered proved successful for removing the toxin melittin (Nat. Commun., 5, 3774, 2014).

Besides toxins derived from metabolic wastes, humans are susceptible to environmental toxins that may enter the body, for instance, by ingestion, absorption through the skin or inhalation. A common well-known toxic metal is lead. For many years, lead was a key component of gasoline in the form of tetraethyl lead and a key component of paints. Currently lead is no longer used or rarely used in these industries, but there are still environmental dangers. Remodeling activities on old homes painted with lead-containing paints produce dusts that may be inhaled or end up in nearby soils, where lead is leached away in ground water or taken up by plants. Unreliable or unregulated water supplies represent a dangerous exposure to Pb²⁺ toxicity, most notably the recent case in Flint, Mich., USA, in which some residents were found to have dangerously high Pb²⁺ levels in their blood after exposure to a new city water supply source. Lead contamination is associated with many ill health effects, including affecting the nervous and urinary systems and inducing learning and developmental disabilities in exposed children. Removal of lead from the blood of afflicted patients would reduce further exposure and damage.

Another well-known toxic metal is mercury. Most human-generated mercury found in the environment comes from the combustion of fossil fuels, the primary source being coal-burning power plants, although various industrial processes also release mercury into the environment. Environmental mercury bioaccumulates in fish and shellfish in the form of methylmercury, which is a highly toxic form of the heavy metal, and consumption of contaminated seafood is the most common cause of mercury poisoning in humans. Once in the body, methyl mercury is likely converted into divalent mercury, where it feeds into a reduction-oxidation pathway. Another common source of exposure is from dental fillings that are composed of mercury amalgams. Elevated blood levels of mercury can cause a wide variety of illnesses including neurological disturbances and renal failure, and these adverse effects are amplified in children.

Chelation therapy is often the preferred treatment of heavy metal poisoning. The chelating agent CaNa₂EDTA (ethylenediamine tetraacetic acid) has been used to remove Pb²⁺ from blood, but this complex is poorly absorbed by the gastrointestinal tract and often must be administered intravenously. It was observed that this chelate could mobilize Pb²⁺, transferring it to other tissues, including the brain (Int. J. Environ. Res. Public Health, 2010, 7, 2745-2788). Dimercaptosuccinic acid (DMSA) was recognized as an antidote for heavy metal poisoning and has been used to treat Pb²⁺ and Hg²⁺ poisoning (See U.S. Pat. No. 5,519,058). Supported chelating agents, i.e., chelating agents bound to resins have been used for heavy metal removal in a dialysis mode, where the blood is on one side of a semi-permeable membrane and the resin-supported chelates on the other side (See U.S. Pat. No. 4,612,122).

Zeolites have been proposed for treating chronic lead poisoning, taken in pill form in US 20180369279A1, but zeolites have limited stability, especially in the gastrointestinal tract.

Applicants have determined that microporous compositions identified as rare earth silicate ion exchange compositions are capable of selectively removing Pb²⁺, Hg²⁺, K⁺ and NH₄⁺ ions from solutions such as bodily fluids or dialysis solutions. Some of the microporous compositions are described in U.S. Pat. No. 6,379,641, which is incorporated by reference. These ion exchangers are further identified by their empirical formulas on an anhydrous basis of:

A^{r+}ₚM^{s+}₁₋ₓM′^{t+}ₓSiₙOₘ

where A is an exchangeable cation such as sodium, M is at least one element selected from the group of rare-earth elements, and M′ is a framework metal having a valence of +2, +3, +4, or +5. Since the compositions are essentially insoluble in bodily fluids (at neutral and mildly acidic or basic pH), they can be orally ingested to remove heavy metal and metabolic toxins from the gastrointestinal system as well as used to remove toxins from dialysis solutions, especially Pb²⁺, Hg²⁺, K⁺ and NH₄⁺.

### SUMMARY OF THE INVENTION

As stated, this invention relates to a process for removing heavy metal and metabolic toxins such as Pb²⁺, Hg²⁺, K⁺, NH₄⁺ or combinations thereof from fluids selected from the group consisting of a bodily fluid, a dialysate solution and mixtures thereof, the process comprising contacting the fluid containing the toxins with a rare-earth silicate ion exchanger at ion exchange conditions thereby removing the toxins from the fluid, the rare-earth silicate ion exchanger having the empirical formula on an anhydrous basis of:

A^{r+}ₚM^{s+}₁₋ₓM′^{t+}ₓSiₙOₘ

In this formula “A” is a structure-directing cation that also serves as a counterbalancing cation and is selected from the group consisting of alkali metals, alkaline earth metals, hydronium ion, ammonium ion, quaternary ammonium ion, and mixtures thereof. Specific examples of alkali metals include, but are not limited to, sodium, potassium and mixtures thereof. Examples of alkaline earth metals include, but are not limited to, magnesium and calcium. “r” is the weighted average valence of A and varies from 1 to 2. The value of “p”, which is the mole ratio of “A” to total metal (total metal=M+M′) varies from about 1 to about 5. The framework structure is composed of silicon, at least one rare-earth element (M) and optionally an M′ metal. The total metal is defined as M+M′, where the mole fraction of total metal that is rare earth metals M is given by “1-x” while the mole fraction of total metal that is M′ metals is given by “x.” The rare-earth elements that are represented by M have a valence of +3 or +4, and include scandium, yttrium, lanthanum, cerium, praseodymium, neodymium, promethium, samarium, europium, gadolinium, terbium, dysprosium, holmium, erbium, thulium, ytterbium, and lutetium. In accordance with these options for M, “s”, the weighted average valence of M, varies from 3 to 4. Similarly, more than one M′ metal can be present and each M′ metal can have a different valence. The M′ metals that can be substituted into the framework have a valence of +2, +3, +4, or +5. Examples of these metals include, but are not limited to, zinc (+2), iron (+3), titanium (+4), zirconium (+4), and niobium (+5). Hence, “t”, the weighted average valence of M′ varies from 2 to 5. Lastly, “n” is the mole ratio of Si to total metal and has a value of about 3 to 10, and “m” is the ratio of O to total metal and is given by

$$m = \frac{[ ( r \cdot p ) + ( s \cdot ( 1 - x ) ) + ( t \cdot x ) + ( 4 \cdot n ) ]}{2}$$

This and other objects and embodiments will become clear after detailed description of the invention.

### DETAILED DESCRIPTION OF THE INVENTION

As stated, applicants have developed a new process for removing toxins from fluids selected from bodily fluids and dialysate solution. One essential element of the instant process is an ion exchanger which has a large capacity and strong affinity, i.e., selectivity for at least one or more heavy metal or metabolic toxins, especially Pb²⁺, Hg²⁺, K⁺ or NH₄⁺. The composition is identified as rare-earth silicate with the composite empirical formula (on an anhydrous basis) of:

A^{r+}ₚM^{s+}₁₋ₓM′^{t+}ₓSiₙOₘ

In this formula “A” is a structure-directing cation that also serves as a counterbalancing cation and is selected from the group consisting of alkali metals, alkaline earth metals, hydronium ion, ammonium ion, quaternary ammonium ion, and mixtures thereof. Specific examples of alkali metals include, but are not limited to, sodium, potassium and mixtures thereof. Examples of alkaline earth metals include, but are not limited to, magnesium and calcium. “r” is the weighted average valence of A and varies from 1 to 2. The value of “p”, which is the mole ratio of “A” to total metal (total metal=M+M′) varies from about 1 to about 5. The framework structure is composed of silicon, at least one rare-earth element (M) and optionally an M′ metal. The total metal is defined as M+M′, where the mole fraction of total metal that is rare earth metals M is given by “1-x” while the mole fraction of total metal that is M′ metals is given by “x.” The rare-earth elements that are represented by M have a valence of +3 or +4, and include scandium, yttrium, lanthanum, cerium, praseodymium, neodymium, promethium, samarium, europium, gadolinium, terbium, dysprosium, holmium, erbium, thulium, ytterbium, and lutetium. In accordance with these options for M, “s”, the weighted average valence of M, varies from 3 to 4. Similarly, more than one M′ metal can be present and each M′ metal can have a different valence. The M′ metals that can be substituted into the framework have a valence of +2, +3, +4, or +5. Examples of these metals include, but are not limited to, zinc (+2), iron (+3), titanium (+4), zirconium (+4), and niobium (+5). Hence, “t”, the weighted average valence of M′ varies from 2 to 5. Lastly, “n” is the mole ratio of Si to total metal and has a value of about 3 to 10, and “m” is the ratio of O to total metal and is given by

$$m = \frac{[ ( r \cdot p ) + ( s \cdot ( 1 - x ) ) + ( t \cdot x ) + ( 4 \cdot n ) ]}{2}$$

The composition has a framework structure that is composed of SiO₂ tetrahedral oxide units, at least one rare-earth metal oxide unit, and optionally an M′ metal oxide unit. Furthermore, the rare-earth metals are 6,7, or 8 coordinate and the M′ metals are 4, 5, or 6 coordinate.

The rare-earth silicates described herein are prepared through hydrothermal crystallization of a reaction mixture prepared by combining reactive sources of silicon, rare-earth metal (M), optionally an M′ metal, at least one cation (A), and water. Silicon sources include, but are not limited to, colloidal silica, fumed silica, tetraorthosilicate, and sodium silicate. Sources of the rare-earth metals (M) include, but are not limited to, metal halides, metal nitrates, metal acetates, metal sulfates, metal oxides, metal hydrous oxides and mixtures thereof. Specific examples of rare-earth metal (M) precursors include, but are not limited to, cerium (III) sulfate, cerium (IV) sulfate, yttrium chloride, ytterbium oxide, ytterbium nitrate, ytterbium sulfate octahydrate, ytterbium carbonate, and ytterbium oxalate. Sources of M′ metals include, but are not limited to, metal halides, metal nitrates, metal acetates, metal oxides, metal hydrous oxide, metal alkoxides, and mixtures thereof. Specific examples include, but are not limited to, zinc chloride, zirconium butoxide, titanium (IV) chloride, titanium (III) chloride solution, niobium (V) chloride, and niobium (V) oxide. Alkali sources include, but are not limited to, sodium hydroxide, potassium hydroxide, rubidium hydroxide, cesium hydroxide, sodium carbonate, potassium carbonate, rubidium carbonate, cesium carbonate, sodium halide, potassium halide, rubidium halide, and cesium halide.

Generally, the hydrothermal process used to prepare the rare-earth silicate ion exchange compositions used in this invention involves forming a reaction mixture containing reactive sources of the required components, which in terms of molar ratios of the oxides is expressed by the following formula:

a A_{2/m}O:1−bMO_{h/2}: b M′O_{g/2}: c SiO₂: d H₂O

where “a” has a value from about 1 to about 100, “m” is the valence of the A components and has values of +1 or +2, “b” has a value from zero to less than 1.0, “h” is the valence of the M components and has values of +3 or +4, “g” is the valence of the M′ components and has values of +2, +3, +4, or +5, “c” has a value of about 0.5 to about 150, and “d” has a value from about 30 to about 10000.

The reaction mixture is prepared by mixing the appropriate sources of rare-earth metal, silicon, templating cation, and optionally an M′ element in any order to give the desired mixture. The basicity of the mixture is controlled by adding excess alkali hydroxide, quaternary ammonium hydroxide, and/or basic compounds of the other constituents of the mixture. The reaction mixture is then reacted at a temperature of about 100° C. to 300° C. for a period of 1 hour to about 30 days in a sealed reaction vessel under autogenous pressure. After the reaction is complete, the resulting mixture is filtered or centrifuged to isolate the solid product, which is washed with deionized water and dried in air or at 100° C. As stated, the compositions of this invention have framework structure of tetrahedral SiO₂ units, at least one rare-earth metal oxide unit, and optionally an M′ metal oxide unit. This framework often results in a microporous structure having an intracrystalline pore system with uniform pore diameters that vary considerably from about 2.5 Å to about 15 Å. On the other hand, the framework of the composition may be layered or amorphous.

As synthesized, the compositions of this invention will contain some of the alkali or alkaline earth metal templating agent in the pores, between layers or in other charge balancing positions. These metals are described as exchangeable cations, meaning that they can be exchanged with other (secondary) A′ cations. Generally, the A exchangeable cations can be exchanged with A′ cations selected from other alkali metal cations (K⁺, Na⁺, Rb^{α}, Cs⁺), alkaline earth cations (Mg²⁺, Ca²⁺, Sr²⁺, Ba²⁺), hydronium ion or mixtures thereof. It is understood that the A′ cation is different from the A cation. The methods used to exchange one cation for another are well known in the art and involve contacting the compositions with a solution containing the desired cation (at molar excess) at exchange conditions. Exchange conditions include a temperature of about 25° C. to about 100° C. and a time of about 20 minutes to about 2 hours. The particular cation (or mixture thereof), which is present in the final product will depend on the particular use of the composition and the specific composition being used. One specific composition is an ion exchanger where the A′ cation is a mixture of Na⁺, Ca²⁺ and H⁺ ions.

As stated above, the materials of this invention are prepared at high pH and as such may increase the pH of any liquid to which they are exposed. Bodily fluids such as gastrointestinal fluids are acidic throughout the digestive tract, reaching pH values as low as 1.0 in the lower stomach. Blood has a pH of about 7.4. Both of these categories of bodily fluids would experience a rise in pH if exposed directly to the as-synthesized materials of this invention. Therefore, it is preferred to ion exchange the materials of this invention. In one preferred embodiment, the as-synthesized rare earth silicate ion-exchanger is treated with acid to form the proton/hydronium exchanged version of the ion-exchanger, which avoids the pH rise on contact with bodily fluids. In another embodiment, the as-synthesized rare earth silicate ion-exchanger may be exchanged with Na⁺ or Ca²⁺ cation or both. In a third embodiment, the as-synthesized rare earth silicate ion-exchanger may be first ion-exchanged with acid before subsequent ion-exchange with Na⁺ or Ca²⁺ or both. If the patient being treated for Pb²⁺ poisoning is hypocalcemic, it will be advantageous to use the Ca²⁺ exchanged form of the rare earth silicate ion-exchanger to avoid reducing Ca²⁺ levels in the patient.

In certain instances, when a quaternary ammonium cation is used in the synthesis, usually as a hydroxide source, the quaternary ammonium cation may be incorporated into the product. Usually, this will not be the case because the quaternary ammonium cations will often be displaced by the alkali cations that have a higher affinity for incorporation into the product. However, the quaternary ammonium ion must be removed from the product. This can often be accomplished by the ion exchange processes mentioned in the previous paragraph. Sometimes the quaternary ammonium ion may be trapped in a pore and it may not be possible to remove the quaternary ammonium cation by ion exchange, in which case a calcination will be required. Typically, the calcination consists of heating the sample to a temperature or 500-600° C. for 2-24 hours in flowing air or in flowing nitrogen followed by flowing air. In this process the quaternary ammonium cation is decomposed and replaced by a residual proton. Once the calcination is completed, the sample can be ion exchanged to the desired A′ cation composition, as described above.

It is also within the scope of the invention that these ion exchange compositions can be used in powder form or can be formed into various shapes by means well known in the art. Examples of these various shapes include pills, extrudates, spheres, pellets and irregularly shaped particles. This has previously been demonstrated in U.S. Pat. No. 6,579,460 B1 and U.S. Pat. No. 6,814,871 B1. The ion exchange compositions of this invention may also be supported, ideally in a porous network including insertion into or binding to a blood compatible porous network such as in a sorption filter as disclosed in U.S. Pat. No. 9,033,908 B2. The porous network may consist of natural or synthetic polymers and biopolymers and mesoporous metal oxides and silicates. Natural polymers (biopolymers) that are suitable may comprise a cross-linked carbohydrate or protein, made of oligomeric and polymeric carbohydrates or proteins. The biopolymer is preferably a polysaccharide. Examples of polysaccharides include α-glucans having 1, 3-, 1, 4- and/or 1, 6-linkages. Among these, the “starch family”, including amylose, amylopectin and dextrins, is especially preferred, but pullulan, elsinan, reuteran and other α-glucans, are also suitable, although the proportion of 1, 6-linkages is preferably below 70%, more preferably below 60%. Other suitable polysaccharides include ß-1, 4-glucans (cellulose), ß-1, 3-glucans, xyloglucans, glucomannans, galactans and galactomannans (guar and locust bean gum), other gums including heterogeneous gums like xanthan, ghatti, carrageenans, alginates, pectin, ß-2, 1- and ß-2, 6-fructans (inulin and Ievan), etc. A preferred cellulose is carboxymethylcellulose (CMC, e. g. AKUCELL from AKZO Nobel). Carbohydrates which can thus be used are carbohydrates consisting only of C, H and O atoms such as, for instance, glucose, fructose, sucrose, maltose, arabinose, mannose, galactose, lactose and oligomers and polymers of these sugars, cellulose, dextrins such as maltodextrin, agarose, amylose, amylopectin and gums, e. g. guar. Preferably, oligomeric carbohydrates with a degree of polymerization (DP) from DP2 on or polymeric carbohydrates from DP50 on are used. These can be naturally occurring polymers such as starch (amylose, amylopectin), cellulose and gums or derivates hereof which can be formed by phosphorylation or oxidation. The starch may be a cationic or anionic modified starch. Examples of suitable (modified) starches that can be modified are corn-starch, potato-starch, rice-starch, tapioca starch, banana starch, and manioc starch. Other polymers can also be used (e. g. caprolactone). In certain embodiments, the biopolymer is preferably a cationic starch, most preferably an oxidized starch (for instance C6 oxidized with hypochlorite). The oxidation level may be freely chosen to suit the application of the sorbent material. Very suitably, the oxidation level is between 5 and 55%, most preferably between 25 and 35%, still more preferably between 28% and 32%. Most preferably the oxidized starch is crosslinked. A preferred crosslinking agent is di-epoxide. The crosslinking level may be freely chosen to suit the application of the sorbent material. Very suitably, the crosslinking level is between 0.1 and 25%, more preferably between 1 and 5%, and most preferably between 2.5 and 3. 5%. Proteins which can be used include albumin, ovalbumin, casein, myosin, actin, globulin, hemoglobin, myoglobin, gelatin and small peptides. In the case of proteins, proteins obtained from hydrolysates of vegetable or animal material can also be used. Particularly preferred protein polymers are gelatin or a derivative of gelatin.

As stated, these compositions have particular utility in adsorbing the metal and metabolic toxins Pb²⁺, Hg²⁺, K⁺ and NH₄⁺ from fluids selected from bodily fluids, dialysate solutions, and mixtures thereof. As used herein and in the claims, bodily fluids will include but not be limited to blood, blood plasma and gastrointestinal fluids. Also, the compositions are meant to be used to treat bodily fluids of any mammalian body, including but not limited to humans, cows, pigs, sheep, monkeys, gorillas, horses, dogs, etc. The instant process is particularly suited for removing toxins from a human body.

There are a number of means for directly or indirectly contacting the fluids with the desired ion exchanger and thus, remove the toxins. One technique is hemoperfusion, which involves packing the above described ion exchange composition into a column through which blood is flowed. One such system is described in U.S. Pat. No. 4,261,828. As stated in the '828 patent, the ion exchange composition is preferably formed into desired shapes such as spheres. Additionally, the ion exchange composition particles can be coated with compounds, such as cellulose derivatives, which are compatible with the blood but nonpermeable for corpuscular blood components. In one specific case, spheres of the desired ion exchange compositions described above can be packed into hollow fibers thereby providing a semipermeable membrane. It should also be pointed out that more than one type of ion-exchange composition can be mixed and used in the process to enhance the efficiency of the process.

Another way of carrying out the process is to prepare a suspension or slurry of the molecular sieve adsorbent by means known in the art such as described is U.S. Pat. No. 5,536,412. The apparatus described in the '412 patent can also be used to carry out the process. The process basically involves passing a fluid, e.g. blood, containing the metal toxins through the interior of a hollow fiber and during said passing, circulating a sorbent suspension against the exterior surfaces of the hollow fiber membrane. At the same time, intermittent pulses of positive pressure are applied to the sorbent solution so that the fluid alternately exits and reenters the interior of the hollow fiber membrane thereby removing toxins from the fluid.

Another type of dialysis is peritoneal dialysis. In peritoneal dialysis, the peritoneal cavity or the abdominal cavity (abdomen) is filled via a catheter inserted into the peritoneal cavity with a dialysate fluid or solution which contacts the peritoneum. Toxins and excess water flow from the blood through the peritoneum, which is a membrane that surrounds the outside of the organs in the abdomen, into the dialysate fluid. The dialysate remains in the body for a time (dwell time) sufficient to remove the toxins. After the required dwell time, the dialysate is removed from the peritoneal cavity through the catheter. There are two types of peritoneal dialysis. In continuous ambulatory peritoneal dialysis (CAPD), dialysis is carried out throughout the day. The process involves maintaining the dialysate solution in the peritoneal cavity and periodically removing the spent dialysate (containing toxins) and refilling the cavity with a fresh dialysate solution. This is carried out several times during the day. The second type is automated peritoneal dialysis or APD. In APD, a dialysate solution is exchanged by a device at night while the patient sleeps. In both types of dialyses, a fresh dialysate solution must be used for each exchange.

The rare-earth silicate ion exchangers of the present invention can be used to regenerate the dialysate solutions used in peritoneal dialysis, thereby further decreasing the amount of dialysate that is needed to cleanse the blood and/or the amount of time needed to carry out the exchange. This regeneration is carried out by any of the means described above for conventional dialysis. For example, in an indirect contacting process, the dialysate from the peritoneal cavity, i.e. first dialysate which has taken up metal toxins transferred across the peritoneum is now contacted with a membrane and a second dialysate solution and metal toxins are transferred across a membrane, thereby purifying the first dialysate solution, i.e. a purified dialysate solution. The second dialysate solution containing the metal toxins is flowed through at least one adsorption bed containing at least one of the ion exchangers described above, thereby removing the metal toxins and yielding a purified second dialysate solution. It is usually preferred to continuously circulate the second dialysate solution through the adsorbent bed until the toxic metal ions have been removed, i.e., Pb²⁺, Hg²⁺, K⁺ or NH₄⁺. It is also preferred that the first dialysate solution be circulated through the peritoneal cavity, thereby increasing the toxic metal removal efficiency and decreasing the total dwell time.

A direct contacting process can also be carried out in which the first dialysate solution is introduced into the peritoneal cavity and then flowed through at least one bed containing at least one ion exchanger. As described above, this can be carried out as CAPD or APD. The composition of the dialysate solution can be varied in order to ensure a proper electrolyte balance in the body. This is well known in the art along with various apparatus for carrying out the dialysis.

The rare-earth silicate ion exchangers can also be formed into pills or other shapes that can be ingested orally and which pick up toxins in the gastrointestinal fluid as the ion exchanger passes through the intestines and is finally excreted. In order to protect the ion exchangers from the high acid content in the stomach, the shaped articles may be coated with various coatings which will not dissolve in the stomach, but dissolve in the intestines.

As has also been stated, although the instant compositions are synthesized with a variety of exchangeable cations (“A”), it is preferred to exchange the cation with secondary cations (A′) which are more compatible with blood or do not adversely affect the blood. For this reason, preferred cations are sodium, calcium, hydronium and magnesium. Preferred compositions are those containing sodium and calcium or sodium, calcium and hydronium ions. The relative amount of sodium and calcium can vary considerably and depends on the composition and the concentration of these ions in the blood.

The x-ray patterns presented in the following examples were obtained using standard x-ray powder diffraction techniques. The radiation source was a high-intensity, x-ray tube operated at 45 kV and 35 mA. The diffraction pattern from the copper K-alpha radiation was obtained by appropriate computer-based techniques. Flat compressed powder samples were continuously scanned at 2° to 70° (2θ). Interplanar spacings (d) in Angstrom units were obtained from the position of the diffraction peaks expressed as θ where θ is the Bragg angle as observed from digitized data. Intensities were determined from the integrated area of diffraction peaks after subtracting background, “Iₒ” being the intensity of the strongest line or peak, and “I” being the intensity of each of the other peaks.

As will be understood by those skilled in the art, the determination of the parameter 20 is subject to both human and mechanical error, which in combination can impose an uncertainty of about ±0.4° on each reported value of 2θ. This uncertainty is, of course, also manifested in the reported values of the d-spacings, which are calculated from the 20 values. This imprecision is general throughout the art and is not sufficient to preclude the differentiation of the present crystalline materials from each other and from the compositions of the prior art. In the x-ray patterns reported, the relative intensities of the d-spacings are indicated by the notations vs, s, m, and w which represent very strong, strong, medium, and weak, respectively. In terms of 100×I/Iₒ, the above designations are defined as:

w>0-15; m>15-60: s>60-80 and vs>80-100

In certain instances, the purity of a synthesized product may be assessed with reference to its x-ray powder diffraction pattern. Thus, for example, if a sample is stated to be pure, it is intended only that the x-ray pattern of the sample is free of lines attributable to crystalline impurities, not that there are no amorphous materials present.

In order to more fully illustrate the instant invention, the following examples are set forth. It is to be understood that the examples are only by way of illustration and are not intended as an undue limitation on the broad scope of the invention as set forth in the appended claims.

### EXAMPLES

### Example 1: Sodium Ytterbium Silicate

In a 250 mL beaker equipped with a high-speed overhead mixer, 9.71 g NaOH pellets (98%) was dissolved in 25.00 g of deionized water. To this solution, 20.25 g colloidal silica (Ludox AS-40, 40% SiO₂) was added and stirred vigorously for 60 minutes. Separately, 5.25 g YbCl₃-6H₂O (99.9%) was dissolved in 125.00 g deionized water that contained 3.75 g concentrated H₂SO₄, yielding a clear solution. The solution containing the digested SiO₂ was then added dropwise to the YbCl₃-6H₂O solution while stirring vigorously using an overhead stirrer at 400RPM, yielding a homogenous white reaction mixture. After stirring for 30 minutes, the reaction mixture was then transferred into 45 cc autoclaves and digested at 200° C. for four days under static conditions. After cooling to room temperature, the product was isolated via centrifugation. The sample was then redispersed in deionized water and then centrifuged again, and this process was repeated two times. The final product was then dried at 100° C. overnight.

Chemical analysis of the product gave an empirical formula of Na_{3.72}YbSi_{7.78}O_{18.93}, and its powder X-ray diffraction pattern was characterized by representative diffraction lines listed in Table 1.

TABLE 1

| 2-Θ | d(Å) | I/I₀ % |
|---|---|---|
| 6.68 | 13.22 | vs |
| 12.88 | 6.87 | m |
| 13.09 | 6.76 | m |
| 13.64 | 6.49 | m |
| 14.99 | 5.91 | m |
| 18.75 | 4.73 | m |
| 19.08 | 4.65 | w |
| 19.27 | 4.60 | m |
| 19.51 | 4.55 | w |
| 21.05 | 4.22 | w |
| 22.09 | 4.02 | w |
| 23.85 | 3.73 | w |
| 24.81 | 3.59 | m |
| 25.79 | 3.45 | m |
| 26.36 | 3.38 | w |
| 26.85 | 3.32 | w |
| 28.06 | 3.18 | w |
| 28.54 | 3.13 | w |
| 29.11 | 3.07 | w |
| 29.73 | 3.00 | m |
| 30.23 | 2.95 | m |
| 30.78 | 2.90 | m |
| 31.15 | 2.87 | w |
| 31.53 | 2.84 | w |
| 33.57 | 2.67 | m |
| 34.58 | 2.59 | w |
| 49.33 | 1.85 | w |
| 52.64 | 1.74 | w |

### Example 2: Sodium Yttrium Silicate

In a 250 mL beaker equipped with a high-speed overhead mixer, 4.85 g NaOH pellets (98%) was dissolved in 12.50 g of deionized water. To this solution, 10.13 g colloidal silica (Ludox AS-40, 40% SiO₂) was added and stirred vigorously for 60 minutes. Separately, 2.59 g Y(NO₃)₃-6H₂O (99.9%) was dissolved in 62.50 g deionized water that contained 1.88 g concentrated H₂SO₄, yielding a clear solution. The solution containing the digested SiO₂ was then added dropwise to the Y(NO₃)₃-6H₂O solution while stirring vigorously using an overhead stirrer at 400RPM, yielding a homogenous white reaction mixture. After stirring for 30 minutes, the reaction mixture was then transferred into 45 cc autoclaves and digested at 200° C. for four days under static conditions. After cooling to room temperature, the product was isolated via centrifugation. The sample was then redispersed in deionized water and then centrifuged again, and this process was repeated two times. The final product was then dried at 100° C. overnight.

Chemical analysis of the product gave an empirical formula of Na_{3.66}YSi_{7.83}O_{18.99}, and its powder X-ray diffraction pattern is characterized by representative diffraction lines listed in Table 2.

TABLE 2

| 2-Θ | d(Å) | I/I₀ % |
|---|---|---|
| 6.69 | 13.21 | vs |
| 12.86 | 6.88 | m |
| 13.09 | 6.76 | m |
| 13.61 | 6.50 | m |
| 14.99 | 5.91 | m |
| 15.20 | 5.82 | w |
| 18.64 | 4.76 | m |
| 19.03 | 4.66 | m |
| 19.25 | 4.61 | m |
| 19.47 | 4.55 | m |
| 21.00 | 4.23 | w |
| 22.06 | 4.03 | w |
| 23.83 | 3.73 | w |
| 24.76 | 3.59 | m |
| 25.74 | 3.46 | w |
| 26.84 | 3.32 | w |
| 28.06 | 3.18 | w |
| 29.11 | 3.07 | w |
| 29.68 | 3.01 | m |
| 30.22 | 2.96 | m |
| 30.68 | 2.91 | m |
| 31.04 | 2.88 | m |
| 31.48 | 2.84 | w |
| 33.54 | 2.67 | m |
| 34.48 | 2.60 | w |
| 34.96 | 2.56 | w |
| 49.31 | 1.85 | w |
| 50.08 | 1.82 | w |
| 52.61 | 1.74 | m |

### Example 3: Sodium Erbium Silicate

In a 250 mL beaker equipped with a high-speed overhead mixer, 5.80 g NaOH pellets (98%) was dissolved in 18.07 g of deionized water. To this solution, 12.16 g colloidal silica (Ludox AS-40, 40% SiO₂) was added and stirred vigorously for 60 minutes. Separately, 3.10 g ErCl₃-6H₂O (99.9%) was dissolved in 78.02 g deionized water that contained 2.25 g concentrated H₂SO₄, yielding a clear solution with a slight red hue. The solution containing the digested SiO₂ was then added dropwise to the ErCl₃-6H₂O solution while stirring vigorously using an overhead stirrer at 400RPM, yielding a homogenous reaction mixture with slight red hue. After stirring for 30 minutes, the reaction mixture was then transferred into 45 cc autoclaves and digested at 200° C. for four days under static conditions. After cooling to room temperature, the product was isolated via centrifugation. The sample was then redispersed in deionized water and then centrifuged again, and this process was repeated two times. The final product was then dried at 100° C. overnight.

Chemical analysis of the product gave an empirical formula of Na_{3.71}ErS_{18.02}O_{21.90}, and its powder X-ray diffraction pattern is characterized by the representative diffraction lines listed in Table 3.

TABLE 3

| 2-Θ | d(Å) | I/I₀ % |
|---|---|---|
| 6.76 | 13.06 | vs |
| 12.82 | 6.90 | m |
| 13.15 | 6.73 | m |
| 13.63 | 6.49 | m |
| 14.14 | 6.26 | w |
| 15.00 | 5.90 | m |
| 18.61 | 4.76 | m |
| 19.00 | 4.67 | m |
| 19.49 | 4.55 | m |
| 19.90 | 4.46 | w |
| 23.85 | 3.73 | w |
| 24.80 | 3.59 | m |
| 25.70 | 3.46 | w |
| 26.88 | 3.31 | w |
| 28.02 | 3.18 | w |
| 29.11 | 3.07 | w |
| 29.67 | 3.01 | m |
| 30.21 | 2.96 | m |
| 30.64 | 2.92 | m |
| 31.16 | 2.87 | w |
| 31.51 | 2.84 | w |
| 33.51 | 2.67 | w |
| 49.23 | 1.85 | m |
| 49.95 | 1.82 | w |
| 52.60 | 1.74 | w |

### Example 4: K⁺-Exchanged Ytterbium Silicate

The product described in this example was synthesized by ion-exchange of Example 1 to yield the potassium form. 2 g of the product described in Example 1 was dispersed in 100 mL of deionized water followed by the addition of 200 mL of 2M KCl solution. The mixture was stirred at 50° C. for 2 hours followed by cooling. The resulting solid was collected by centrifugation and the process was repeated two more times. The final product was washed three times and dried overnight at 100° C.

The powder X-ray diffraction pattern of the product is characterized by representative diffraction lines shown in Table 4.

TABLE 4

| 2-Θ | d(Å) | I/I₀ % |
|---|---|---|
| 6.80 | 12.99 | s |
| 7.18 | 12.30 | m |
| 12.78 | 6.92 | m |
| 13.67 | 6.47 | m |
| 14.76 | 6.00 | w |
| 18.72 | 4.74 | w |
| 20.11 | 4.41 | m |
| 23.98 | 3.71 | w |
| 25.62 | 3.47 | w |
| 27.66 | 3.22 | w |
| 29.34 | 3.04 | m |
| 30.24 | 2.95 | m |
| 31.34 | 2.85 | w |
| 32.84 | 2.72 | w |
| 47.86 | 1.90 | w |
| 49.68 | 1.83 | w |
| 49.78 | 1.83 | w |
| 52.51 | 1.74 | w |
| 54.85 | 1.67 | w |
| 62.35 | 1.49 | w |

### Example 5: K⁺-Exchanged Yttrium Silicate

The product described in this example was synthesized by ion-exchange of Example 2 to yield the potassium form. 2 g of the product described in Example 2 was dispersed in 100 mL of deionized water followed by the addition of 200 mL of 2M KCl solution. The mixture was stirred at 50° C. for 2 hours followed by cooling. The resulting solid was collected by centrifugation and the process was repeated two more times. The final product was washed three times and dried overnight at 100° C.

Chemical analysis of the product gave an empirical formula of K_{2.65}YSi_{5.72}O_{14.27}, and its powder X-ray diffraction pattern is characterized by the representative diffraction lines listed in Table 5.

TABLE 5

| 2-Θ | d(Å) | I/I₀ % |
|---|---|---|
| 6.92 | 12.76 | s |
| 12.79 | 6.91 | m |
| 13.65 | 6.48 | m |
| 14.67 | 6.04 | m |
| 18.83 | 4.71 | m |
| 20.15 | 4.40 | m |
| 24.00 | 3.71 | m |
| 25.72 | 3.46 | m |
| 29.33 | 3.04 | s |
| 30.30 | 2.95 | s |
| 31.25 | 2.86 | m |
| 31.99 | 2.80 | w |
| 32.80 | 2.73 | m |
| 49.40 | 1.84 | m |
| 52.48 | 1.74 | w |

### Example 6: Tin-Doped Sodium Ytterbium Silicate

A tin-doped version of Example 1 was prepared as follows. In a 250 mL beaker equipped with a high-speed overhead mixer, 6.45 g NaOH pellets (98%) was dissolved in 20.13 g of deionized water. To this solution, 13.49 g colloidal silica (Ludox AS-40, 40% SiO₂) was added and stirred vigorously for 60 minutes. Separately, 3.19 g YbCl₃-6H₂O (99.9%) was dissolved in 80.10 g deionized water that contained 2.43 g concentrated H₂SO₄, yielding a clear solution. The solution containing the digested SiO₂ was then added dropwise to the YbCl₃-6H₂O solution while stirring vigorously using an overhead stirrer at 400RPM, yielding a homogenous white reaction mixture. After stirring for 1 hour, 0.18 g SnCl₄-5H₂O was added and the reaction solution was stirred for an additional 1 hour. The resulting reaction mixture was then transferred into 45 cc autoclaves and digested at 200° C. for four days under static conditions. After cooling to room temperature, the product was isolated via centrifugation. The sample was then redispersed in deionized water and then centrifuged again, and this process was repeated two times. The final product was then dried at 100° C. overnight.

An analysis of the product using a scanning electron microscope equipped with energy dispersive X-ray spectroscopy showed a homogeneous distribution of Sn in material. Chemical analysis of the product gave an empirical formula of Na_{5.00}Yb_{0.73}Sn_{0.27}Si_{7.68}O_{19.50}, and its powder X-ray diffraction pattern is characterized by the representative diffraction lines listed in Table 6.

TABLE 6

| 2-Θ | d(Å) | I/I₀ % |
|---|---|---|
| 6.99 | 12.64 | vs |
| 13.06 | 6.78 | s |
| 13.90 | 6.37 | m |
| 15.17 | 5.84 | m |
| 15.52 | 5.71 | m |
| 19.09 | 4.64 | m |
| 19.46 | 4.56 | m |
| 20.05 | 4.42 | w |
| 22.41 | 3.97 | m |
| 25.11 | 3.54 | m |
| 26.06 | 3.42 | m |
| 28.41 | 3.14 | w |
| 30.18 | 2.96 | m |
| 30.62 | 2.92 | vs |
| 30.95 | 2.89 | vs |
| 31.59 | 2.83 | m |
| 32.19 | 2.78 | w |
| 33.88 | 2.64 | w |
| 50.06 | 1.82 | w |
| 52.98 | 1.73 | w |

### Example 7: Potassium Ytterbium Silicate

In 250 mL beaker equipped with a high-speed overhead mixer, 16.07 g KOH pellets (86%) was dissolved in 26.37 g of deionized water. To this solution, 42.93 g of colloidal silica (Ludox AS-30, 30% SiO₂) was added and stirred vigorously for 30 minutes. Separately, a second solution was prepared by dissolving 5.29 g of YbCl_{3.6}H₂O (99%) in 8.33 g of deionized water, which was then added dropwise while stirring. The reaction mixture was stirred vigorously for 2.5 hours and then transferred to a high-speed blender, where it was homogenized for 1 minute. The mixture was then transferred into 45 cc autoclaves and digested at 200° C. for five days under static conditions. After cooling to room temperature, the product was isolated via centrifugation, washed with deionized water, and then dried at 100° C. overnight.

Chemical analysis of the product gave an empirical formula of K_{3.67}YbSi_{7.89}O_{19.11}, and its powder X-ray diffraction pattern was characterized by the data presented in Table 7.

TABLE 7

| 2Θ | d(Å) | I/I₀ % |
|---|---|---|
| 5.83 | 15.14 | vs |
| 9.86 | 8.96 | m |
| 13.03 | 6.79 | w |
| 13.45 | 6.58 | m |
| 15.29 | 5.80 | m |
| 15.59 | 5.68 | m |
| 15.97 | 5.55 | m |
| 17.00 | 5.21 | m |
| 23.31 | 3.81 | m |
| 24.70 | 3.60 | m |
| 25.90 | 3.44 | m |
| 27.52 | 3.24 | m |
| 28.40 | 3.14 | w |
| 29.25 | 3.05 | w |
| 30.56 | 2.93 | m |
| 31.33 | 2.85 | m |
| 32.16 | 2.78 | w |
| 33.50 | 2.67 | m |

### Example 8: Sodium Cerium Silicate

In a 250 mL beaker equipped with a high-speed overhead mixer, 19.41 g NaOH pellets (98%) were dissolved in 50.50 g of deionized water. To this solution, 40.51 g colloidal silica (Ludox AS-40, 40% SiO₂) was added and stirred vigorously for 60 minutes. Separately, 8.98 g Ce(SO₄)₂ (99.9%) was dissolved in 250.40 g deionized water that contained 7.50 g concentrated H₂SO₄, yielding a bright orange solution. The solution containing the digested SiO₂ was then added dropwise to the Ce(SO₄)₂ solution while stirring vigorously using an overhead stirrer at 400RPM, yielding a homogenous white reaction mixture. After stirring for 60 minutes, the reaction mixture was then transferred into 45 cc autoclaves and digested at 200° C. for four days under static conditions. After cooling to room temperature, the product was isolated via centrifugation, washed with deionized water, and then dried at 100° C. overnight.

The oxidation state of Ce in the resulting product was analyzed using X-ray absorption near edge spectroscopy (XANES), which indicated essentially all the cerium atoms are in the +4 oxidation state (Ce⁴⁺). Chemical analysis of the product gave an empirical formula of Na_{1.24}CeSi_{3.68}O_{9.98}, and its powder X-ray diffraction pattern was characterized by the data presented in Table 8.

TABLE 8

| 2Θ | d(Å) | I/I₀ % |
|---|---|---|
| 11.96 | 7.40 | m |
| 12.41 | 7.13 | w |
| 13.53 | 6.54 | vs |
| 13.77 | 6.42 | m |
| 17.24 | 5.14 | m |
| 18.38 | 4.82 | w |
| 21.12 | 4.20 | w |
| 21.71 | 4.09 | m |
| 23.99 | 3.71 | w |
| 25.08 | 3.55 | m |
| 27.20 | 3.28 | m |
| 27.72 | 3.22 | m |
| 28.09 | 3.17 | m |
| 28.44 | 3.14 | m |
| 29.99 | 2.98 | w |
| 30.35 | 2.94 | w |
| 32.96 | 2.72 | w |
| 34.86 | 2.57 | w |
| 41.42 | 2.18 | w |
| 42.96 | 2.10 | w |
| 45.45 | 1.99 | w |
| 46.13 | 1.97 | w |

### Example 9: NH₄⁺-Exchanged Cerium Silicate

The product described in the following example was synthesized by ion-exchange of Example 9 to yield the ammonium form. 3 g of the product described in Example 9 was dispersed in 250 mL of 2M NH₄Cl exchange solution. Three ion-exchanges were performed at 50° C. for 2 hours each step. The exchanged solid was isolated using centrifugation, washed with deionized water, and then dried at 100° C. overnight. The powder X-ray diffraction pattern of the product is characterized by representative diffraction lines shown in Table 9.

TABLE 9

| 2-Θ | d(Å) | I/I₀ % |
|---|---|---|
| 11.68 | 7.57 | s |
| 12.16 | 7.27 | w |
| 13.50 | 6.56 | s |
| 16.90 | 5.24 | m |
| 18.22 | 4.87 | w |
| 20.88 | 4.25 | w |
| 21.43 | 4.14 | m |
| 23.52 | 3.78 | w |
| 24.72 | 3.60 | m |
| 26.56 | 3.35 | m |
| 27.20 | 3.28 | m |
| 28.05 | 3.18 | m |
| 29.89 | 2.99 | w |
| 32.03 | 2.79 | w |
| 32.75 | 2.73 | w |
| 34.34 | 2.61 | m |
| 34.86 | 2.57 | w |
| 35.63 | 2.52 | w |
| 36.42 | 2.47 | w |
| 36.90 | 2.43 | w |
| 37.53 | 2.39 | w |
| 38.71 | 2.32 | w |
| 42.49 | 2.13 | w |
| 43.22 | 2.09 | w |
| 43.70 | 2.07 | w |
| 45.49 | 1.99 | m |
| 48.16 | 1.89 | w |
| 48.62 | 1.87 | w |

### Example 10: Removal of Pb²⁺ and Hg²⁺ Ions from Solution

The samples disclosed in Examples 1-9 were tested to determine their ability to selectively adsorb Pb²⁺ and Hg²⁺ ions from a solution that also contained essential electrolytes found in the body, including Na, K, Mg, and Ca. The test solutions were prepared by dissolving sodium nitrate, potassium nitrate, magnesium nitrate, calcium nitrate, and lead (or mercury) nitrate in a sodium acetate buffer solution. The buffer solution was used to maintain a constant pH of approximately 4.7, and 1 L of buffer solution was prepared by dissolving 4.18 g sodium acetate and 2.49 g acetic acid in 1 L of deionized water. The test solutions were first analyzed by ICP and contained approximate concentrations of 3000 ppm Na⁺, 300 ppm 25 ppm Mg²⁺, 25 ppm Ca²⁺, and 200 ppb Pb²⁺ (or 200 ppb Hg²⁺). For the test, 100 mg of the rare-earth silicate ion exchanger was placed in a 125 mL plastic bottle along with 100 mL of the testing solution. The capped bottles were tumbled at room temperature for 2 hours. Once the ion-exchanger has been in contact with the test solution for the desired amount of time, the solid/solution suspension is passed through a 0.2 μm syringe filter to remove the solids, and then the solution is analyzed using ICP. The K_{d} value for the distribution of metals between solution and solid was calculated using the following formula:

$${K}_{d} ( mL \text{/g} ) = \frac{( V ) ( Ac )}{( W ) ( Sc )} 1$$

where: V=volume of waste simulant (mL)
Ac=concentration of cation absorbed on ion-exchanger (g/mL)
W=mass of ion-exchanger evaluated (g)
Sc=concentration of cation in post reaction supernatant (g/mL)
   Table 10 and Table 11 below summarize the results of the Pb²⁺ and Hg²⁺ uptake studies, respectively. The data left blank in the tables indicate no statistical change in electrolyte concentration or an increase in concentration due to the release of cations from the rare-earth ion exchanger. The criterion for including an ion-exchanger in this application is it had to remove at least 75% of the heavy metal (Pb²⁺, Hg²⁺), while simultaneously not removing more than 10% of the other electrolytes in the test solution.

TABLE 10
Pb²⁺, Na⁺, K⁺, Mg²⁺, Ca²⁺
uptake expressed as K_{d} values (mL/g).

| Example | Pb²⁺ K_{d} | Na⁺ K_{d} | K⁺ K_{d} | Mg²⁺ K_{d} | Ca²⁺ K_{d} |
|---|---|---|---|---|---|
| 1 | 15,637 | — | 28 | 26 | — |
| 2 | 21,651 | — | 78 | — | — |
| 3 | 46,000 | — | 55 | — | — |
| 4 | 46,000 | 19 | — | 30 | 12 |
| 5 | 37,367 | 10 | — | 21 | — |
| 6 | 31,787 | — | 41 | 16 | 8 |
| 7 | 19,971 | — | — | 4 | — |
| 9 | 15,593 | 7 | 40 | — | 9 |
| 10 | 19,364 | 7 | 36 | — | 9 |

TABLE 11
Hg²⁺, Na⁺, K⁺, Mg²⁺, Ca²⁺
uptake expressed as K_{d} values (mL/g).

| Example | Hg²⁺ K_{d} | Na⁺ K_{d} | K⁺ K_{d} | Mg²⁺ K_{d} | Ca²⁺ K_{d} |
|---|---|---|---|---|---|
| 1 | 5,844 | — | 41 | 4 | — |
| 2 | 3,610 | — | 41 | 4 | — |
| 3 | 2,983 | — | 73 | — | — |
| 4 | 3,201 | — | — | 4 | — |
| 7 | 4,296 | — | — | 29 | 21 |

### Example 12: Removal of K⁺ and NH₄⁺ Ions from Solution

The samples disclosed in Examples 1-9 were tested to determine their ability to selectively adsorb K⁺ and NH₄⁺ ions from a simulated dialysate solution that contained essential electrolytes found in the body, including Mg, and Ca. The test solutions were prepared by dissolving sodium chloride, potassium chloride, calcium chloride dihydrate, magnesium chloride hexahydrate, and ammonium chloride in 1 L of a 40 mM (mM=millimolar) sodium bicarbonate solution. The test solutions were first analyzed by aqueous cation liquid chromatography and contained approximate concentrations of 507 ppm NH₄⁺, 109 ppm K⁺, 3053 ppm Na⁺, 37 ppm Ca²⁺, and 9.5 ppm Mg²⁺. For the test, 100 mg of the rare-earth silicate ion exchanger was placed in a 20 mL plastic vial along with 20 mL of the dialysate solution. The vials were then tumbled at room temperature for 2 hours. Once the ion-exchanger has been in contact with the test solution for the desired amount of time, the solid/solution suspension is passed through a 0.2 μm syringe filter to remove the solids, and then the solution was analyzed using aqueous liquid chromatography.

Table 11 and Table 12 summarize the results of the uptake studies, showing the change in cation concentration (expressed in ppm) and the amount of cation absorbed by each material on a mmol/gram basis, respectively.

TABLE 11
NH₄⁺, K⁺, Mg²⁺, Ca²⁺ uptake summary

| Test | 506.9 ppm | 108.9 ppm | 9.5 ppm | 37.3 ppm |
|---|---|---|---|---|
| Solution: | NH₄⁺ | K⁺ | Mg²⁺ | Ca²⁺ |
| Example | NH₄⁺ (ppm) | K⁺ (ppm) | Mg²⁺ (ppm) | Ca²⁺ (ppm) |
| 2 | 469.3 | 90.0 | 9.4 | 36.1 |
| 8 | 467.9 | 19.2 | 9.3 | 32.0 |

TABLE 12
NH₄⁺, K⁺, Mg²⁺, Ca²⁺ uptake in
mmol of cation per gram of material

| | NH₄⁺ | K⁺ | Mg²⁺ | Ca²⁺ |
|---|---|---|---|---|
| Example | (mmol/g) | (mmol/g) | (mmol/g) | (mmol/g) |
| 2 | 0.42 | 0.10 | 0.001 | 0.01 |
| 8 | 0.43 | 0.46 | 0.002 | 0.03 |

### Specific Embodiments

While the following is described in conjunction with specific embodiments, it will be understood that this description is intended to illustrate and not limit the scope of the preceding description and the appended claims.

A first embodiment of the invention is a process for removing Pb²⁺, Hg²⁺, K⁺ and NH₄⁺ toxins or mixtures thereof from bodily fluids comprising contacting the fluid containing the toxins with an ion exchanger to remove the toxins from the fluid by ion exchange between the ion exchanger and the bodily fluid, the ion exchanger being a rare-earth silicate composition with an empirical formula on an anhydrous basis of A^{r+}ₚM^{s+}₁₋ₓM′^{t+}ₓSiₙOₘ where A is an exchangeable cation selected from the group consisting of alkali metals, alkaline earth metals, hydronium ion, ammonium ion, quaternary ammonium ion and mixtures thereof, “r” is the weighted average valence of A and varies from 1 to 2, “p” is the mole ratio of A to total metal (total metal=M+M′) and varies from about 1 to about 5, “M” is a framework rare earth metal selected from the group consisting of scandium, yttrium, lanthanum, cerium, praseodymium, neodymium, promethium, samarium, europium, gadolinium, terbium, dysprosium, holmium, erbium, thulium, ytterbium, and lutetium and mixtures thereof, “s” is the weighted average valence of M and varies from 3 to 4, “1-x” is the mole fraction of total metal that is M, M′ is a framework metal having a valence of +2, +3, +4, or +5, “t” is the weighted average valence of M′ and varies from 2 to 5, “x” is the mole fraction of total metal that is M′ and varies from 0 to 0.99, “n” is the mole ratio of Si to total metal and has a value of about 3 to about 10, and “m” is the mole ratio of O to total metal and is given by

$$m = \frac{[ ( r \cdot p ) + ( s \cdot ( 1 - x ) ) + ( t \cdot x ) + ( 4 \cdot n ) ]}{2} .$$

An embodiment of the invention is one, any or all of prior embodiments in this paragraph up through the first embodiment in this paragraph wherein the bodily fluid is selected from the group consisting of whole blood, blood plasma, or other component of blood, gastrointestinal fluids and dialysate solution containing blood, blood plasma, other component of blood or gastrointestinal fluids. An embodiment of the invention is one, any or all of prior embodiments in this paragraph up through the first embodiment in this paragraph where x=0. An embodiment of the invention is one, any or all of prior embodiments in this paragraph up through the first embodiment in this paragraph where A is a mixture of calcium and an alkali metal. An embodiment of the invention is one, any or all of prior embodiments in this paragraph up through the first embodiment in this paragraph where A is not potassium. An embodiment of the invention is one, any or all of prior embodiments in this paragraph up through the first embodiment in this paragraph where A is not ammonium. An embodiment of the invention is one, any or all of prior embodiments in this paragraph up through the first embodiment in this paragraph where the ion exchanger is packed into hollow fibers incorporated into a membrane. An embodiment of the invention is one, any or all of prior embodiments in this paragraph up through the first embodiment in this paragraph wherein the ion exchanger is contained on particles coated with a coating comprising a cellulose derivative composition. An embodiment of the invention is one, any or all of prior embodiments in this paragraph up through the first embodiment in this paragraph wherein the process is a hemoperfusion process wherein the bodily fluid is passed through a column containing the ion exchanger. An embodiment of the invention is one, any or all of prior embodiments in this paragraph up through the first embodiment in this paragraph wherein a dialysate solution is introduced into a peritoneal cavity and then is flowed through at least one adsorbent bed containing at least one of the ion exchanger. An embodiment of the invention is one, any or all of prior embodiments in this paragraph up through the first embodiment in this paragraph wherein the ion exchanger is formed into a shaped article to be ingested orally, followed by ion exchange between the ion exchanger and the Pb²⁺, Hg²⁺, K⁺ and NH₄⁺ toxins contained in a gastrointestinal fluid in a mammal's intestines and then by excretion of the ion exchanger containing the toxins. An embodiment of the invention is one, any or all of prior embodiments in this paragraph up through the first embodiment in this paragraph wherein the shaped article is coated with a coating that is not dissolved by conditions within a stomach.

A second embodiment of the invention is a composition comprising a combination of a bodily fluid, a dialysate solution or a mixture of the bodily fluid and the dialysate solution the combination further comprising a rare earth silicate ion exchanger having an empirical formula on an anhydrous basis of A^{r+}ₚM^{s+}₁₋ₓM′^{t+}ₓSiₙOₘ where A is an exchangeable cation selected from the group consisting of alkali metals, alkaline earth metals, hydronium ion, ammonium ion, quaternary ammonium ion and mixtures thereof, “r” is the weighted average valence of A and varies from 1 to 2, “p” is the mole ratio of A to total metal (total metal=M+M′) and varies from about 1 to about 5, “M” is a framework rare earth metal selected from the group consisting of scandium, yttrium, lanthanum, cerium, praseodymium, neodymium, promethium, samarium, europium, gadolinium, terbium, dysprosium, holmium, erbium, thulium, ytterbium, and lutetium and mixtures thereof, “s” is the weighted average valence of M and varies from 3 to 4, “1-x” is the mole fraction of total metal that is M, M′ is a framework metal having a valence of +2, +3, +4, or +5, “t” is the weighted average valence of M′ and varies from 2 to 5, “x” is the mole fraction of total metal that is M′ and varies from 0 to 0.99, “n” is the mole ratio of Si to total metal and has a value of about 3 to about 10, and “m” is the mole ratio of O to total metal and is given by

$$m = \frac{[ ( r \cdot p ) + ( s \cdot ( 1 - x ) ) + ( t \cdot x ) + ( 4 \cdot n ) ]}{2} .$$

An embodiment of the invention is one, any or all of prior embodiments in this paragraph up through the second embodiment in this paragraph wherein the bodily fluid is whole blood, blood plasma, other blood component or gastrointestinal fluid.

A third embodiment of the invention is an apparatus comprising a matrix containing a support material for a rare earth silicate ion exchanger having an empirical formula on an anhydrous basis of A^{r+}ₚM^{s+}₁₋ₓM′^{t+}ₓSiₙOₘ where A is an exchangeable cation selected from the group consisting of alkali metals, alkaline earth metals, hydronium ion, ammonium ion, quaternary ammonium ion and mixtures thereof, “r” is the weighted average valence of A and varies from 1 to 2, “p” is the mole ratio of A to total metal (total metal=M+M′) and varies from about 1 to about 5, “M” is a framework rare earth metal selected from the group consisting of scandium, yttrium, lanthanum, cerium, praseodymium, neodymium, promethium, samarium, europium, gadolinium, terbium, dysprosium, holmium, erbium, thulium, ytterbium, and lutetium and mixtures thereof, “s” is the weighted average valence of M and varies from 3 to 4, “1-x” is the mole fraction of total metal that is M, M′ is a framework metal having a valence of +2, +3, +4, or +5, “t” is the weighted average valence of M′ and varies from 2 to 5, “x” is the mole fraction of total metal that is M′ and varies from 0 to 0.99, “n” is the mole ratio of Si to total metal and has a value of about 3 to about 10, and “m” is the mole ratio of O to total metal and is given by

$$m = \frac{[ ( r \cdot p ) + ( s \cdot ( 1 - x ) ) + ( t \cdot x ) + ( 4 \cdot n ) ]}{2} .$$

An embodiment of the invention is one, any or all of prior embodiments in this paragraph up through the third embodiment in this paragraph wherein the matrix comprises a porous network comprising biocompatible polymers and metal oxides and silicates. An embodiment of the invention is one, any or all of prior embodiments in this paragraph up through the third embodiment in this paragraph wherein the biocompatible polymers comprise cross-linked carbohydrates or proteins. An embodiment of the invention is one, any or all of prior embodiments in this paragraph up through the third embodiment in this paragraph wherein the biocompatible polymer is a polysaccharide selected from α-glucans having 1, 3-, 1, 4- or 1, 6 linkages. An embodiment of the invention is one, any or all of prior embodiments in this paragraph up through the third embodiment in this paragraph wherein the biocompatible polymer is a carbohydrate selected from glucose, fructose, sucrose, maltose, arabinose, mannose, galactose, lactose and oligomers and polymers comprising one or more of the carbohydrates. An embodiment of the invention is one, any or all of prior embodiments in this paragraph up through the third embodiment in this paragraph wherein the biocompatible polymer comprises a protein selected from albumin, ovalbumin, casein, myosin, actin, globulin, hemoglobin, myoglobin, gelatin and small peptides.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions.

The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

We claim as our invention:

1. A process for removing Pb²⁺, Hg²⁺, K⁺ and NH₄⁺ toxins or mixtures thereof from bodily fluids comprising contacting the fluid containing the toxins with an ion exchanger to remove the toxins from the fluid by ion exchange between the ion exchanger and the bodily fluid, the ion exchanger being a rare-earth silicate composition with an empirical formula on an anhydrous basis of:
A^{r+}ₚM^{s+}₁₋ₓM′^{t+}ₓSiₙOₘ
where A is an exchangeable cation selected from the group consisting of alkali metals, alkaline earth metals, hydronium ion, ammonium ion, quaternary ammonium ion and mixtures thereof, “r” is the weighted average valence of A and varies from 1 to 2, “p” is the mole ratio of A to total metal (total metal=M+M′) and varies from about 1 to about 5, “M” is a framework rare earth metal selected from the group consisting of scandium, yttrium, lanthanum, cerium, praseodymium, neodymium, promethium, samarium, europium, gadolinium, terbium, dysprosium, holmium, erbium, thulium, ytterbium, and lutetium and mixtures thereof, “s” is the weighted average valence of M and varies from 3 to 4, “1-x” is the mole fraction of total metal that is M, M′ is a framework metal having a valence of +2, +3, +4, or +5, “t” is the weighted average valence of M′ and varies from 2 to 5, “x” is the mole fraction of total metal that is M′ and varies from 0 to 0.99, “n” is the mole ratio of Si to total metal and has a value of about 3 to about 10, and “m” is the mole ratio of O to total metal and is given by
$$m = \frac{[ ( r \cdot p ) + ( s \cdot ( 1 - x ) ) + ( t \cdot x ) + ( 4 \cdot n ) ]}{2} .$$

2. The process of claim 1 wherein the bodily fluid is selected from the group consisting of whole blood, blood plasma, or other component of blood, gastrointestinal fluids and dialysate solution containing blood, blood plasma, other component of blood or gastrointestinal fluids.

3. The process of claim 1 where x=0.

4. The process of claim 1 where A is a mixture of calcium and an alkali metal.

5. The process of claim 1 where A is not potassium.

6. The process of claim 1 where A is not ammonium.

7. The process of claim 1 where the ion exchanger is packed into hollow fibers incorporated into a membrane.

8. The process of claim 1 wherein said ion exchanger is contained on particles coated with a coating comprising a cellulose derivative composition.

9. The process of claim 1 wherein said process is a hemoperfusion process wherein said bodily fluid is passed through a column containing said ion exchanger.

10. The process of claim 1 wherein a dialysate solution is introduced into a peritoneal cavity and then is flowed through at least one adsorbent bed containing at least one of said ion exchanger.

11. The process of claim 1 wherein said ion exchanger is formed into a shaped article to be ingested orally, followed by ion exchange between said ion exchanger and said Pb²⁺, Hg²⁺, K⁺ and NH₄⁺ toxins contained in a gastrointestinal fluid in a mammal's intestines and then by excretion of said ion exchanger containing said toxins.

12. The process of claim 11 wherein said shaped article is coated with a coating that is not dissolved by conditions within a stomach.

13. A composition comprising a combination of a bodily fluid, a dialysate solution or a mixture of said bodily fluid and said dialysate solution said combination further comprising a rare earth silicate ion exchanger having an empirical formula on an anhydrous basis of:
A^{r+}ₚM^{s+}₁₋ₓM′^{t+}ₓSiₙOₘ
where A is an exchangeable cation selected from the group consisting of alkali metals, alkaline earth metals, hydronium ion, ammonium ion, quaternary ammonium ion and mixtures thereof, “r” is the weighted average valence of A and varies from 1 to 2, “p” is the mole ratio of A to total metal (total metal=M+M′) and varies from about 1 to about 5, “M” is a framework rare earth metal selected from the group consisting of scandium, yttrium, lanthanum, cerium, praseodymium, neodymium, promethium, samarium, europium, gadolinium, terbium, dysprosium, holmium, erbium, thulium, ytterbium, and lutetium and mixtures thereof, “s” is the weighted average valence of M and varies from 3 to 4, “1-x” is the mole fraction of total metal that is M, M′ is a framework metal having a valence of +2, +3, +4, or +5, “t” is the weighted average valence of M′ and varies from 2 to 5, “x” is the mole fraction of total metal that is M′ and varies from 0 to 0.99, “n” is the mole ratio of Si to total metal and has a value of about 3 to about 10, and “m” is the mole ratio of O to total metal and is given by
$$m = \frac{[ ( r \cdot p ) + ( s \cdot ( 1 - x ) ) + ( t \cdot x ) + ( 4 \cdot n ) ]}{2} .$$

14. The composition of claim 13 wherein said bodily fluid is whole blood, blood plasma, other blood component or gastrointestinal fluid.

15. An apparatus comprising a matrix containing a support material for a rare earth silicate ion exchanger having an empirical formula on an anhydrous basis of:
A^{r+}ₚM^{s+}₁₋ₓM′^{t+}ₓSiₙOₘ
where A is an exchangeable cation selected from the group consisting of alkali metals, alkaline earth metals, hydronium ion, ammonium ion, quaternary ammonium ion and mixtures thereof, “r” is the weighted average valence of A and varies from 1 to 2, “p” is the mole ratio of A to total metal (total metal=M+M′) and varies from about 1 to about 5, “M” is a framework rare earth metal selected from the group consisting of scandium, yttrium, lanthanum, cerium, praseodymium, neodymium, promethium, samarium, europium, gadolinium, terbium, dysprosium, holmium, erbium, thulium, ytterbium, and lutetium and mixtures thereof, “s” is the weighted average valence of M and varies from 3 to 4, “1-x” is the mole fraction of total metal that is M, M′ is a framework metal having a valence of +2, +3, +4, or +5, “t” is the weighted average valence of M′ and varies from 2 to 5, “x” is the mole fraction of total metal that is M′ and varies from 0 to 0.99, “n” is the mole ratio of Si to total metal and has a value of about 3 to about 10, and “m” is the mole ratio of O to total metal and is given by
$$m = \frac{[ ( r \cdot p ) + ( s \cdot ( 1 - x ) ) + ( t \cdot x ) + ( 4 \cdot n ) ]}{2} .$$

16. The apparatus of claim 15 wherein said matrix comprises a porous network comprising biocompatible polymers and metal oxides and silicates.

17. The apparatus of claim 16 wherein said biocompatible polymers comprise cross-linked carbohydrates or proteins.

18. The apparatus of claim 16 wherein said biocompatible polymer is a polysaccharide selected from α-glucans having 1, 3-, 1, 4- or 1, 6 linkages.

19. The apparatus of claim 16 wherein said biocompatible polymer is a carbohydrate selected from glucose, fructose, sucrose, maltose, arabinose, mannose, galactose, lactose and oligomers and polymers comprising one or more of said carbohydrates.

20. The apparatus of claim 16 wherein said biocompatible polymer comprises a protein selected from albumin, ovalbumin, casein, myosin, actin, globulin, hemoglobin, myoglobin, gelatin and small peptides.
